(19) United States
(12) Patent Application Publication
(10) Pub. No.: US 2023/0285254 A1
(43) Pub. Date: Sep. 14, 2023

(54) HAIR CONDITIONING COMPOSITION

(71) Applicant: The Procter & Gamble Company, Cincinnati, OH (US)

(72) Inventor: Gemma Zoe BRAGANZA, Singapore (SG)

(21) Appl. No.: 18/179,486

(22) Filed: Mar. 7, 2023

Related U.S. Application Data
(60) Provisional application No. 63/318,424, filed on Mar. 10, 2022.

(51) Int. Cl. A61K 8/34; A61Q 5/12; A61K 8/42

(52) U.S. Cl. CPC A61K 8/342; A61Q 5/12; A61K 8/42; A61K 2800/34; A61K 2800/31

(57) ABSTRACT

An anhydrous hair care composition comprising from about 5% to about 50% of a cationic surfactant; a fatty alcohol; and at least about 40% of a solvent system comprising a mono-alcohol.

### FIELD OF THE INVENTION

The present invention relates to an anhydrous hair conditioning composition.

### BACKGROUND OF THE INVENTION

Consumers are looking to reduce their environmental impact. Typically, hair care products are provided in disposable plastic containers, which have a high perceived environmental impact. Refills can be purchased that have a lower volume of plastic, however this solution may still not meet the environmental concerns from the consumer. The most sustainable solutions are often waterless solid products, which do not typically have good performance when used.

Concentrated forms may provide a hair care solution that can reduce the environmental impact. Typical concentrated hair care product, however, would have a high viscosity and be hard for the consumer to dilute into a functionable and homogenous finished product.

Thus, there remains an unmet need for a highly concentrated form of hair conditioner that can be diluted easily with tap water in the consumer's home, and that is instantly usable and has good performance.

### SUMMARY OF THE INVENTION

The present invention is directed to a hair care composition comprising:
a. from about 5% to about 50% of a cationic surfactant;
b. a fatty alcohol; and
c. at least about 40% of a solvent system comprising a mono-alcohol;
   wherein the composition is anhydrous.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Q.S. herein means up to 100%.

### Composition

The present invention provides a super concentrate that can be diluted by the consumer in their home with tap water in a durable and re-usable dispensing container. The benefits of this technology are:
Low viscosity but high concentration that makes it easy to mix in the consumer's home
Easily forms a hair conditioning product upon dilution with water
High efficacy comparable to currently marketed products
Is anhydrous so requires less plastic packaging than typical conditioner products by not including the water
Is associated with environmental and sustainable benefits that are important to many consumers.

The present invention provides a highly concentrated anhydrous form of hair conditioner which can be diluted easily with tap water in the consumer's home due to the conditioner's low viscosity nature. The product made is instantly usable and has good performance. By determining the right combination of hair conditioning materials and solvent system, this low viscosity concentrate can be achieved. Although a clear low viscosity liquid can be formed with concentrated conditioner actives, many will not form a gel phase with increased viscosity upon mixing with water.

The concentrated conditioner is anhydrous, clear, and has a very low viscosity, similar to water. It instantly forms a conditioner gel network when mixed with an aqueous phase or water, which is a very surprising and unique transformation.

The clear concentrate can also contain perfumes, preservatives, silicones, oils, colourants etc. to additionally enhance the conditioning properties and consumer desirability.

### A. Solvent System

The selection of solvents is critical. Although a clear low viscosity liquid can be formed with concentrated conditioner actives, many will not form a gel phase with increased viscosity upon mixing with water.

The solvent system may comprise a mono-alcohol. Suitable examples include, without being limited to, ethanol, isopropanol, butanol, hexanol, heptanol, dodecanol, oleyl alcohol and combinations thereof. Also possible as a solvent may be a sugar alcohol. Suitable examples of sugar alcohol could be from the diol class and may be, without being limited to, propylene glycol, dipropylene glycol, pentylene glycol, hexylene glycol, and combinations thereof.

The total composition may comprise at least about 40%, by weight of the composition, of the solvent system. In some embodiments, the conditioner composition may comprise from about 40% to about 95%, by weight, of the solvent system. In other embodiments, the composition may comprise from about 45%, about 50%, about 55%, or about 60% to about 90%, about 85%, about 80%, about 75%, about 70%, or about 65%, by weight of the composition, of the solvent system. When both a mono-alcohol and sugar alcohol are used, mono-alcohol may make up at least 10% by weight of the composition. In some embodiments, the mono-alcohol may be linear or branched.

In some cases, the absence of or too little mono-alcohol in the formulation can cause the composition to not form a single phase. For example, in Comparative Examples C2, C5, C6, and C7, all comprise less than 40% of a mono-alcohol or mono-alcohol plus sugar alcohol combined. And in Comparative Examples C3 and C4, no mono-alcohol is used, only sugar alcohol. And in C2 to C7, each composition formed is not a single phase. All the inventive compositions herein provide a single phase. If the anhydrous, concentrated conditioner has a solid precipitate out, i.e., is a solid-liquid dispersion, when water is added, the solid will not quickly form a gel network. The hydrated composition would likely have solid lumps in a cream, which would not be desirable to consumers.

Similarly, if solvents other than a mono-alcohol or a mono-alcohol plus a sugar alcohol are used, a single phase anhydrous concentrated composition may form, but it may not be able to form a gel network with the addition of water, such as in Comparative Examples C8, C9, and C10. The mono-alcohols used may be liquid at about 25° C. or 30° C.

Without being bound, it is the Inventors' belief that use of the particular solvents described herein are critical, as the solvent system should have an affinity and solubility in water and also an ability to solubilize the gel network. Mono-alcohol and glycols are miscible with water, vs. for example, benzyl alcohol, which can form a clear low viscosity concentrate, but itself is not miscible with water (low solubility in water).

### B. Cationic Surfactant System

The hair care composition described herein may comprise a cationic surfactant system. The cationic surfactant system can be one cationic surfactant or a mixture of two or more cationic surfactants. Suitable cationic surfactants may include, for example, behentrimonium methosulfate (BTMS), behentrimonium chloride (BTMAC), stearamidopropyldimethylamine (SAPDMA), behenamidopropyldimethylamine (BAPDMA), brassicyl valinate esylate, and combinations thereof. Other possible cationic surfactants may include brassicyl valinate esylate, or brassicamidopropyl dimethylanamine.

The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system can be included in the hair care composition at a level of from about 5% to about 50%, in some embodiments from about 6%, about 7%, about 8%, about 9%, or about 10% to about 40%, from about 20% to about 40%, from about 30% to about 50%, or from about 30% to about 50%, by weight of the hair care composition.

### Mono-Long Alkyl Quaternized Ammonium Salt

The monoalkyl quaternized ammonium salt cationic surfactants useful herein are those having one long alkyl chain which has from 12 to 30 carbon atoms, from 16 to 24 carbon atoms, and in one embodiment at C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

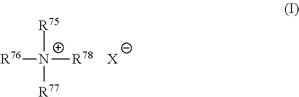

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, an/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as [symbol] -glutamic acid, lactic acid, hydrochloric acid, malic acid, succinic acid, acetic acid, fumaric acid, tartaric acid, citric acid, [symbol] -glutamic hydrochloride, maleic acid, mandelic acid, and mixtures thereof. The amines herein can be partially neutralized with any of the acids at a molar ratio of the amine to the acid of from about 1:0.3 to about 1:2, and/or from about 1:0.4 to about 1:1.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The dialkyl quaternized ammonium salt cationic surfactants useful herein are those having two long alkyl chains having 12-30 carbon atoms, and/or 16-24 carbon atoms, and/or 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

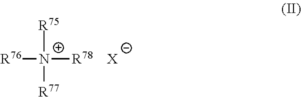

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, and/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### C. Fatty Alcohol

The fatty alcohol can be included in the composition at a level of from about 1%, to about 50% by weight of the composition, in view of providing the benefits of the present invention, in some embodiments from about 5% to about 40, by weight of the composition.

The fatty alcohol useful herein is selected from the group consisting of fatty alcohols, fatty alcohol derivatives, fatty acid derivatives, and mixtures thereof. It is understood by the artisan that the alcohols disclosed in this section of the specification can in some instances fall into more than one classification, e.g., some fatty alcohol derivatives can also be classified as fatty acid derivatives. However, a given classification is not intended to be a limitation on that particular alcohol but is done so for convenience of classification and nomenclature. Nonlimiting examples of the alcohols are found in International Cosmetic Ingredient Dictionary, Fifth Edition, 1993, and CTFA Cosmetic Ingredient Handbook, Second Edition, 1992.

The fatty alcohols useful herein are those having from about 10 to about 30 carbon atoms, preferably from about 16 to about 22 carbon atoms. These fatty alcohols can be saturated or unsaturated, and can be straight or branched chain alcohols.

Preferred fatty alcohols include, for example, cetyl alcohol, stearyl alcohol, behenyl alcohol, myristyl alcohol, lauryl alcohol, brassica alcohol and mixtures thereof. These alcohols are known to form stable gel network when combined with cationic surfactants. In the present invention, more preferred fatty alcohols are cetyl alcohol, stearyl alcohol and mixtures thereof.

Together with a fatty alcohol, the compositions can further comprise a low melting fatty alcohol, for example, oleyl alcohol.

### D. Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.1% to about 30%, preferably up to about 20% by weight of the composition.

### Silicone Compound

The compositions of the present invention may, or may not, contain a silicone compound. It is believed that the silicone compound can provide smoothness and softness on dry hair. The silicone compounds herein can be used at levels by weight of the composition of preferably from about 0.1% to about 30%, more preferably from about 5% to about 20%, still more preferably from about 10% to about 20%.

The silicone compounds useful herein, as a single compound, as a blend or mixture of at least two silicone compounds, or as a blend or mixture of at least one silicone compound and at least one solvent, have a viscosity of preferably from about 50 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferred polyalkyl siloxanes include, for example, polydimethylsiloxane, polydiethylsiloxane, and polymethylphenylsiloxane. Polydimethylsiloxane, which is also known as dimethicone, is especially preferred. These silicone compounds are available, for example, from the General Electric Company in their Viscasil® and TSF 451 series, and from Dow Corning in their Dow Corning SH₂₀₀ series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

Silicone compounds useful herein also include amino substituted materials. Preferred aminosilicones include, for example, those which conform to the general formula (I):

(R₁)ₐG₃₋ₐ—Si—(—OSiG₂)ₙ—(—OSiG_{b}(R₁)_{2-b})ₘ—O—SiG₃₋ₐ(R₁)ₐ
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH_{2.} Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning. Some embodiments may include Silicone Quaternium-26.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben, imidazolidinyl urea, p-Hydroxyacetophenone; pH adjusting agents, such as citric acid, benzoic acid, sorbic acid, sodium citrate, succinic acid, phosphoric acid, sodium hydroxide, sodium carbonate; coloring agents, such as any of the FD&C or D&C dyes; perfumes; and sequestering agents, such as disodium ethylenediamine tetra-acetate; ultraviolet and infrared screening and absorbing agents such as benzophenones; and antidandruff agents such as zinc pyrithione, piroctone olamine. In some embodiments, the composition may be free of any silicone, sulfate, and/or paraben. The composition may further comprise a material selected from the group consisting of perfumes, preservatives, silicones, colorants, oils, acidifier, and combinations thereof.

### Low Melting Point Oil

The compositions may comprise one or more conditioning oils. Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils including pentaerythritol tetraisostearate, trimethylol ester oils, citrate ester oils, and glyceryl ester oils; poly α-olefin oils such as polydecenes; and mixtures thereof. Additional oils may include polyester oil or mono-, di, tri-ether or ester including triglycerides, such as caprylic capric triglyceride or vegetable oils such as coconut oil, soybean oil, rapeseed oil, cocoa butter, olive oil, palm oil, rice bran oil, and mixtures thereof.

In some embodiments, a conditioning oil may have a hydrophilic—lipophilic balance (HLB) of less than about 10. In some embodiments, the oil may be a mono, di, or tri ester or ether where the monomer units have a carbon chain of C2 to C16, preferably C4 to C10, or more preferably C6 to C8. In some embodiments, the oil may be a polyester with the hydrophobic monomer units (linear or branched) having carbon chains shorter than C16, preferably shorter than C12. Commercially available oil examples include, but are not limited to, Myritol 318 from BASF (caprylic/capric triglyceride), Plantasil Micro from BASF (dicaprylyl ether in emulsion form (Dicaprylyl Ether (and) Decyl Glucoside (and) Glyceryl Oleate)); or Citropol 1A from P2 science (Polycitronellol Acetate).

### Anhydrous

The present invention provides a highly concentrated anhydrous form of conditioner that can be diluted easily with tap water in the consumer's home. The concentrated conditioner may be anhydrous, clear, and very low viscosity, similar to the viscosity of water. The present inventive hair care composition instantly forms a conditioner gel network when mixed with an aqueous phase or water.

Anhydrous herein means that the composition comprises at most 2% total water and no added water, meaning at most 2% bound water.

### Product Forms and Method of Use

The hair care composition may be a low viscosity liquid. The liquid may be a single homogenous phase. The consumer may mix water with the inventive hair care composition in a ratio from about 1:1 to about 1:20 to form a thick cream or gel, which may have a shear stress of at least about 100 Pa when measured on a rheometer at a shear rate of 950 s⁻¹. This hydrated conditioner may form a gel network and then be used in any known manner as a hair conditioner.

The inventive hair care composition, before water is added, may be clear or transparent. To be transparent or clear is defined as a percent transmittance (% T) of at least 80% transmittance at 600 nm, according to the Clarity Assessment Test Method herein. Upon the addition of water, the composition may no longer be transparent.

The inventive composition may have water added to it to form a hair conditioning composition. The amount of water added can vary but may be from about 1:1 to about 1:20 ratio of the anhydrous concentrate to water. In some embodiments, the ratio of anhydrous concentrate to water may be from about 1:1 to about 1:6. The hair conditioning composition then may have a shear stress of at least about 100 Pa at a shear rate of 950 s⁻¹.

A method of making a hair conditioner, comprising the following steps:
a. forming an anhydrous composition comprising:
   (1) from about 5% to about 50% of a cationic surfactant;
   (2) a fatty alcohol; and
   (3) at least about 40% of a solvent system comprising a mono-alcohol;
b. adding water to the composition in the ratio of composition to water of 1:1 to 1:20;
wherein the hair conditioner has a shear stress of at least 100 Pa at a shear rate of 950 s⁻¹.
   A method of making a hair conditioner, comprising the following steps:
a. forming an anhydrous composition comprising:
   (1) from about 5% to about 50% of a cationic surfactant;
   (2) a fatty alcohol; and
   (3) at least about 40% of a solvent system comprising a mono-alcohol;
b. adding water to the composition in the ratio of composition to water of 1:1 to 1:20;
wherein the hair conditioner forms a gel network.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Invention Examples

Inventive examples A and B and 1-10 are anhydrous, single phase compositions having a viscosity less than 100 cPs. When mixed with water in a ratio between 1:1 and 1:20, a lamellar gel network is formed. The existence of the lamellar gel network matrix with a L_{β} phase is shown by the X-15 ray (SAX/WAX) analysis data. For Inventive Examples A and B, the gel network is shown by the SAXS d-spacing and WXRD reflection.

Both WXRD and SAXS data are generated as described in more detail below in the Test Methods. As can be seen in Table 1, Inventive Examples A and B, when mixed with water at 1:4.7 (1 part Inventive Example to 4.7 parts water), exhibit SAXS d-spacings corresponding to the interspacing ratios of lamella phases and also WXRD reflection in the 3.5 to 4.5 range, indicating a lamellar gel network matrix. Comparative Examples from Table 3, in contrast, exhibit phase separation upon mixing with water, which indicates that the lamellar gel network matrix with a L_{β} phase cannot form without the specified solvent combination.

TABLE 1

| Example | | | | | | | | | | | | |
|---|---|---|---|---|---|---|---|---|---|---|---|---|
| Formulation | A | B | 1 | 2 | 3 | 4 | 5 | 6 | 7 | 8 | 9 | 10 |
| SAPDMA | 15.6 | 7.2 | 15.5 | 15.5 | 7.75 | 15.5 | 7.75 | 15.5 | 7.75 | 15.5 | 7.75 | 15.5 |
| Cetyl Alcohol | 20.6 | 9.5 | 20.5 | 20.5 | 10.25 | 20.5 | 10.25 | 20.5 | 10.25 | 20.5 | 10.25 | 20.5 |
| Stearyl Alcohol | 14.1 | 6.5 | 9 | 9 | 4.5 | 9 | 4.5 | 9 | 4.5 | 9 | 4.5 | 9 |
| Salicylic Acid | 5 | 2.5 | 5 | 5 | 2.5 | 5 | 2.5 | 5 | 2.5 | 5 | 2.5 | 5 |
| Ethanol | 32 | 35.2 | | 50 | 75 | | | 25 | 37.5 | 25 | 37.5 | |
| IPA | | | 50 | | | 25 | 37.5 | | | | | 37.5 |
| Dipropylene glycol | 12.7 | 34.9 | | | | 25 | 37.5 | 25 | 37.5 | | | 12.5 |
| Pentylene glycol | | | | | | | | | | 25 | 37.5 | |
| Other inclusions | | 4.2 | | | | | | | | | | |
| SAXS d-spacing | 50.7, | 56.7, | | | | | | | | | | |
| (nm) d₁, d₂,d₃,d₄, d₅ | 25.2, | 28.5, | | | | | | | | | | |
| | 16.9, | 18.8, | | | | | | | | | | |
| | 12.6, | 14.1, | | | | | | | | | | |
| | 10.1 | 11.4 | | | | | | | | | | |
| WXRD (Å) | 4.1 | 4.1 | | | | | | | | | | |

### Comparative Examples

Comparative Examples C2, C5, C6, and C7 all comprise less than 40% of a mono-alcohol or mono-alcohol plus sugar alcohol combined and do not form a single phase fluid. In Comparative Examples C3 and C4, no mono-alcohol is used, only sugar alcohol. In C2 to C7, each composition formed is not a single phase liquid, meaning they do not have a viscosity less than 100 Pa and there is a solid precipitate.

TABLE 2

| Example | | | | | | |
|---|---|---|---|---|---|---|
| Formulation | C2 | C3 | C4 | C5 | C6 | C7 |
| SAPDMA | 23.25 | 23.25 | 7.75 | 23.25 | 23.25 | 23.25 |
| Cetyl Alcohol | 30.75 | 30.75 | 10.25 | 30.75 | 30.75 | 30.75 |
| Stearyl Alcohol | 13.5 | 13.5 | 4.5 | 13.5 | 13.5 | 13.5 |
| Salicylic Acid | 7.5 | 7.5 | 2.5 | 7.5 | 7.5 | 7.5 |
| Ethanol | 25 | | | | 12.5 | 12.5 |
| IPA | | | | 12.5 | | |
| Dipropylene glycol | | 25 | 75 | 12.5 | 12.5 | |
| Pentylene glycol | | | | | | 12.5 |

Table 3 shows Comparative Examples C8, C9, and C10, compositions without at least about 40% solvent system comprising a mono-alcohol. Comparative Examples C8, C9 and C10 each form a low viscosity liquid, however, they do not form a gel network structure when mixed with water. Also, none have a shear stress of at least 100Pa at a shear rate of 950 s⁻¹ when the composition is combined with water in a ratio of composition to water from 1:1 to 1:20.

TABLE 3

| Example | | | |
|---|---|---|---|
| Formulation | C8 | C9 | C10 |
| SAPDMA | 7.75 | 15.5 | 9.3 |
| Cetyl | 10.25 | 20.5 | 12.3 |
| Alchol | | | |
| Stearyl | 7 | 14 | 8.4 |
| Alcohol | | | |
| Salicylic | 2.5 | 5 | 3 |
| Acid | | | |
| Dipropylene | | | |
| glycol | | | |
| Pentylene | | 25 | |
| glycol | | | |
| Phenoxy | | | |
| Ethanol | | | |
| Perfume- | 75 | | |
| RL | | | |
| Benzyl | | 25 | 70 |
| Alcohol | | | |

### Test Methods

### SAX/WAXS Analysis Method

Small-angle x-ray scattering (“SAXS”) as used to resolve periodic structures in mesophases is essentially an x-ray diffraction technique. It is used in conjunction with conventional wide-angle x-ray diffraction (“WXRD”) to characterize aggregate structures such as micelles, gel networks, lamella, hexagonal and cubic liquid crystals. The different mesophases that show periodic structures can be characterized by the relative positions (d-spacing) of their reflections as derived from the Bragg equation (d=λ/2 Sin θ) where d represents the interplanar spacing, λ the radiation wavelength and θ the scattering (diffraction) angle.

The one dimensional lamella gel network phase is characterized by the ratio of the interplanar spacings d₁/d₁, d₁/d₂, d₁/d₃, d₁/d₄, d₁/d₅ having the values 1:2:3:4:5 etc. in the SAXS region (long-range order) and one or two invariant reflection(s) in the WXRD region (short-range) centered around 3.5 and 4.5 Å over a broad halo background. Other mesophases (e.g. hexagonal or cubic) will have characteristically different d-spacing ratios.

The SAXS data is collected in high resolution collimation for 60 minutes on a Xenocs Xeuss 2.0 with Dectris Pilatus 3R 200K-A detector at a sample to detector distance of 2500.00 mm. The setup has an evacuated chamber. The specimen is injected into a quartz-glass capillary tube (diameter=2.0 mm, length=80 mm, wall thickness=0.01 mm), mounted in grooved capillary sample holder and placed in the path of the x-ray beam. Data are collected and analyzed using the Xenocs Foxtrot software. The 2-D data are azimuthally integrated and reduced to intensity versus scattering vector (ω) or its d equivalent by the SAXS utilities software.

The WAXS data is collected using a Bruker X-Ray Diffraction D8 ADVANCE equipped with LYNXEYE XET detector. The specimen is loaded to a 20 mm PMMA sample holder for measurements at ambient conditions, and 2theta scan is performed in the range of 20°-23° using CuKα radiation (1.54060 Å). The voltage and current applied are 40 kV and 40 mA respectively. Each measurement is performed with a step size of 0.02° and 1 s per step.

### Rheology and Viscosity Method

Rheology and viscosity are used to evaluate and characterize product samples. The key rheology methods identified are mentioned below:
Shear Stress at 950 s⁻¹ via flow curve: This is the method to ramp up shear rate logarithmically from 0.1 to 1000 s⁻¹ in 1 min using a cone & plate geometry, and to read the shear stress value σ (Pa) at shear rate 950 s⁻¹.
Viscosity, measured is centipoise, cPs, by a Cone Plate Viscometer, such as Brookfield Viscometer RV-DV2TRV available from Brookfield Engineering Laboratories (USA) or other substitutable model known in the art. A cone such as CPA-41Z rotated at 1 rpm with sample volume 2 ml with a 3-minute measurement duration with the measurement read at the end of this 3 minutes. The exact selection of measurement conditions may be selected as needed by one skilled in the art.

For the inventive compositions, the viscosity may be below 100 cPs. For inventive compositions, when combined with water in a ratio of composition to water from about 1:1 to about 1:20, the shear stress may be at least 100 Pa at a shear rate of 950 s⁻¹.

### Clarity Assessment—Measurement of % Transmittance (% T)

Composition clarity can be measured by % Transmittance. For this composition to be clear, the composition may be made without ingredients that would give the composition a hazy appearance such as silicones, opacifiers, non-silicone oils, micas, and gums. It is believed that adding these ingredients would not impact the nature of the compositions function, however these ingredients may obscure measurement of clarity by % Transmittance. A clear composition will generally be a single phase, and should remain clear even when shaken, while two-phase compositions will not be clear when shaken.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. An example of equipment used for measurement is X-Rite Ci7800 Bench Top Spectrophotometer. The compositions of the present invention may have a percent transmittance (% T) of at least about 80% transmittance at 600 nm.

### Examples/Combinations

A. A hair care composition comprising:
a. from about 5% to about 50% of a cationic surfactant;
b. a fatty alcohol; and
c. at least about 40% of a solvent system comprising a mono-alcohol;
   wherein the composition is anhydrous.
      B. The composition of paragraph A, wherein the mono-alcohol is selected from the group consisting of ethanol, isopropanol, butanol, hexanol, heptanol, dodecanol, oleyl alcohol and combinations thereof.
      C. The composition of any one of paragraphs A or B, wherein the solvent system further comprises a sugar alcohol.
      D. The composition of paragraph C, wherein the sugar alcohol is selected from the group consisting of propylene glycol, dipropylene glycol, penylene glycol, hexylene glycol, and combinations thereof.
      E. The composition of paragraph C, wherein the ratio of mono-alcohol to sugar alcohol is from about 99:1 to about 50:50, by weight.
      F. The composition of any one of paragraphs A to E, wherein the composition comprises at least about 50%, by weight of the composition, of the solvent system.
      G. The composition of any one of paragraphs A to F, wherein the composition further comprises a quaternizing acid.
      H. The composition of paragraph G, wherein the quaternizing acid is selected from the group consisting of [symbol] -glutamic acid, salicylic acid, succinic acid, citric acid, benzoic acid, lactic acid, malic acid, succinic acid, acetic acid, fumaric acid, tartaric acid, maleic acid, mandelic acid and combinations thereof.
      I. The composition of any one of paragraphs A to H, wherein the cationic surfactant is selected from the group consisting of SAPDMA, BAPDMA, and combinations thereof.
      J. The composition of any one of paragraphs A to I, wherein the composition comprises from about 5% to about 40%, by weight of the composition, of cationic surfactant.
      K. The composition of any one of paragraphs A to J, wherein the composition further comprises an additional surfactant.
      L. The composition of any one of the preceding claims, wherein the composition comprises from about 1% to about 50%, by weight of the composition, of fatty alcohol.
      M. The composition of any one of paragraphs A to L, wherein the composition is free of silicone.
      N. The composition of any one of paragraphs A to M, wherein the composition is free of any sulfate and/or paraben.
      O. The composition of any one of paragraphs A to N, wherein the composition is transparent and has a viscosity below 100 cP.
      P. The composition of any one of paragraphs A to O, wherein the composition further comprises a material selected from the group consisting of perfumes, preservatives, silicones, colorants, oils, acidifier, and combinations thereof.
      Q. The composition of any one of paragraphs A to P, wherein the composition is a single phase.
      R. A method of making a hair conditioner, comprising the following steps:
a. forming an anhydrous composition comprising:
   (1) from about 5% to about 50% of a cationic surfactant;
   (2) a fatty alcohol; and
   (3) at least about 40% of a solvent system comprising a mono-alcohol;
b. adding water to the composition in the ratio of composition to water of 1:1 to 1:20;
wherein the hair conditioner has a shear stress of at least 100 Pa at a shear rate of 950 s⁻¹.
   S. The method of paragraph R, wherein the hair conditioner forms a gel network.
   T. A method of making a hair conditioner, comprising the following steps:
a. forming an anhydrous composition comprising:
   (1) from about 5% to about 50% of a cationic surfactant;
   (2) a fatty alcohol; and
   (3) at least about 40% of a solvent system comprising a mono-alcohol;
b. adding water to the composition in the ratio of composition to water of 1:1 to 1:20;
wherein the hair conditioner forms a gel network.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a. from about 5% to about 50% of a cationic surfactant;
b. a fatty alcohol; and
c. at least about 40% of a solvent system comprising a mono-alcohol;
wherein the composition is anhydrous.

2. The composition of claim 1, wherein the mono-alcohol is selected from the group consisting of ethanol, isopropanol, butanol, hexanol, heptanol, dodecanol, oleyl alcohol and combinations thereof.

3. The composition of claim 1, wherein the solvent system further comprises a sugar alcohol.

4. The composition of claim 3, wherein the sugar alcohol is selected from the group consisting of propylene glycol, dipropylene glycol, penylene glycol, hexylene glycol, and combinations thereof.

5. The composition of claim 3, wherein the ratio of mono-alcohol to sugar alcohol is from about 99:1 to about 50:50, by weight.

6. The composition of claim 1, wherein the composition comprises at least about 50%, by weight of the composition, of the solvent system.

7. The composition of claim 1, wherein the composition further comprises a quaternizing acid.

8. The composition of claim 7, wherein the quaternizing acid is selected from the group consisting of [symbol] -glutamic acid, salicylic acid, succinic acid, citric acid, benzoic acid, lactic acid, malic acid, succinic acid, acetic acid, fumaric acid, tartaric acid, maleic acid, mandelic acid and combinations thereof.

9. The composition of claim 1, wherein the cationic surfactant is selected from the group consisting of SAPDMA, BAPDMA, and combinations thereof.

10. The composition of claim 1, wherein the composition comprises from about 5% to about 40%, by weight of the composition, of cationic surfactant.

11. The composition of claim 1, wherein the composition further comprises an additional surfactant.

12. The composition of claim 1, wherein the composition comprises from about 1% to about 50%, by weight of the composition, of fatty alcohol.

13. The composition of claim 1, wherein the composition is free of silicone.

14. The composition of claim 1, wherein the composition is free of any sulfate and/or paraben.

15. The composition of claim 1, wherein the composition is transparent and has a viscosity below 100 cP.

16. The composition of claim 1, wherein the composition further comprises a material selected from the group consisting of perfumes, preservatives, silicones, colorants, oils, acidifier, and combinations thereof.

17. The composition of claim 1, wherein the composition is a single phase.

18. A method of making a hair conditioner, comprising the following steps:
a. forming an anhydrous composition comprising:
(1) from about 5% to about 50% of a cationic surfactant;
(2) a fatty alcohol; and
(3) at least about 40% of a solvent system comprising a mono-alcohol;
b. adding water to the composition in the ratio of composition to water of 1:1 to 1:20;
wherein the hair conditioner has a shear stress of at least 100 Pa at a shear rate of 950 s⁻¹.

19. The method of claim 18, wherein the hair conditioner forms a gel network.

20. A method of making a hair conditioner, comprising the following steps:
a. forming an anhydrous composition comprising:
(1) from about 5% to about 50% of a cationic surfactant;
(2) a fatty alcohol; and
(3) at least about 40% of a solvent system comprising a mono-alcohol;
b. adding water to the composition in the ratio of composition to water of 1:1 to 1:20;
wherein the hair conditioner forms a gel network.
